(19) United States
(12) Patent Application Publication
(10) Pub. No.: US 2007/0059828 A1
(43) Pub. Date: Mar. 15, 2007

(54) Carrier for nucleic acid molecule delivery

(76) Inventors: Tetsuji Yamaoka, Osaka (JP); Yutaka Tokiwa, Ibaraki (JP); Masaru Kitagawa, Shiga (JP)

Correspondence Address: FISH & RICHARDSON P.C., P.O. BOX 1022, MINNEAPOLIS, MN, 55440-1022, US

(21) Appl. No.: 10/578,283

(22) PCT Filed: Nov. 5, 2004
(86) PCT No.: PCT/JP04/16447

§ 371 Date: May 4, 2006

(30) Foreign Application Priority Data
Nov. 7, 2003 (JP) 2003-379096
May 7, 2004 (JP) 2004-138734

(51) Int. Cl. C12N 15/00; C08G 63/91

(52) U.S. Cl. 435/455; 525/54.2

(57) ABSTRACT

A carrier for nucleic acid molecule delivery formed from a saccharified copolymer comprising repeating unit (A) having a cationic group, saccharified repeating unit (B). There is further provided a carrier for nucleic acid molecule delivery formed from a saccharified copolymer comprising, in addition to the repeating units (A) and (B), repeating unit (C) having a hydrophobic substituent.

### TECHNICAL FIELD

The present invention relates to a carrier for nucleic acid molecule delivery formed from a saccharified copolymer.

### BACKGROUND ART

In a gene therapy which has recently attracted attention, “ex vivo gene transfection” where cells removed from a body of a patient are transfected with a foreign gene in a culture system, transformed cells are grown and then transplanted again in the patient is mainly carried out. In this case, since the cells capable of being isolated from the patient are limited, peripheral blood lymphocytes are used in many cases. However, the cells to be targeted are different depending on subjected diseases, and in particular, when somatic cells or cells in organ tissue are targeted, it is necessary to directly administer a plasmid DNA encoding a foreign gene to the body (in vivo gene transfection).

The in vivo gene transfection has been studied by many researchers, and it has been strongly desired to develop a carrier for nucleic acid molecule delivery which can efficiently introduce the gene into the body and assure safety.

Meanwhile, adenovirus and retrovirus have been used in 85% or more of gene therapy protocols approved by US FDA (Annu. Rev. Microbiol., 49, 807, 1995). The gene transfection with the virus is highly effective and significant, but troubles considered to be caused by immure response to the virus have occurred in the gene therapy carried out in USA, and a risk such as virus replication has been pointed out.

Thus, it has been studied to use non-viral carriers such as cationic liposomes (Proc. Natl. Acad. Sci. USA, 84, 7413, 1987) and cation polymers (Proc. Natl. Acad. Sci. USA, 92, 7297, 1995; Bioconjugate Chem., 6, 7, 1995) as the carrier for nucleic acid molecule delivery. Specifically, linear polycations such as diethylaminoethyl dextran (DEAE-dex) and poly-L-lysine (PLL) have been studied conventionally.

A gene expression efficiency of the polycation is lower than that of the liposome, but the polycation is advantageous in that the polycation has a lower propensity to accumulate in liver than the liposome when directly administered in the body and is relatively easily controlled in its pharmacodynamics. However, poly-L-lysine which is one of the polycations and have been studied conventionally and energetically as the carrier for nucleic acid molecule delivery has a low efficiency in gene transfection ability.

In the light of such problems, the present inventors invented a technology concerning a saccharified copolymer having a particular structure and a carrier for nucleic acid molecule delivery formed from the saccharified copolymer, and previously filed the patent application (JP 2004-26866 A), but have been demanded to study on more enhancement of the gene expression efficiency.

### DISCLOSURE OF INVENTION

### Problems to be Solved by the Invention

The present invention mainly aims at providing a carrier for nucleic acid molecule delivery formed from a saccharified copolymer, which is excellent in gene expression efficiency, releases the nucleic acid when introduced into cells and exhibits a high expression efficiency of the nucleic acid.

### MEANS FOR SOLVING THE PROBLEMS

As a result of an extensive study on a carrier for nucleic acid molecule delivery capable of effectively expressing a gene, the present inventors have found that the carrier for nucleic acid molecule delivery which exhibits excellent effects is obtained when a saccharified copolymer having a particular structure is used, and have completed the present invention by further studying extensively.

That is, the present invention pertains to the following carriers for nucleic acid molecule delivery.

1. A saccharified copolymer having a repeating unit (A) having a cationic group, a repeating unit (B) containing sugar and a repeating unit (C) having a hydrophobic substituent.

2. The saccharified copolymer according to item 1 wherein the repeating unit (A) having the cationic group is represented by the general formula (I): 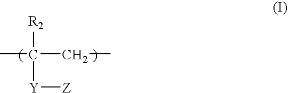
wherein R₂ represents H or CH₃; Y represents —C(═O)O—(CH₂)n_{y}-, —OC(═O)— (CH₂)n_{y}-, —OC(═O)— (CH₂)n_{y}-C(═O) or —CONH— (CH₂)n_{y}- and n_{y} represents an integer of 1 to 10; and Z represents —NR₃R₄ (R₃ and R₄ are the same or different and represent hydrocarbon groups having 1 to 10 carbon atoms), —N⁺R₅R₆R₇ (R₅, R₆ and R₇ are the same or different and represent hydrocarbon groups having 1 to 10 carbon atoms) or a nitrogen-containing heterocyclic group.

3. The carrier for nucleic acid molecule delivery according to item 1 wherein the repeating unit (B) containing the sugar is represented by the general formula (II): 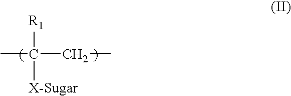
wherein R₁ represents —H or —CH₃; Sugar represents a sugar residue obtained by removing one NH₂ from a sugar (in the cases of monosaccharide, disaccharide or polysaccharide where the sugar is bound at an amino group in amino sugar) or removing one OH from a sugar (in the cases of monosaccharide, disaccharide or polysaccharide where the sugar is bound at a hydroxyl group in saccharide); and X represents —C(═O)Z¹, —C(═O)O-Rₐ—, —CONH—, —CONH—R_{b}—, —OC(═O)—R_{c}—C(═O)Z¹ or —Ph—R_{d}-Z¹ wherein Rₐ represents —Ph—O— or —(CH₂)nₐ-O— and nₐ represents an integer of 1 to 10, R_{b} represents —Ph—O— or —(CH₂)n_{b}-O— and n_{b} represents an integer of 1 to 10, R_{c} represents —(CH₂)n_{c}- or —(CH₂)n_{c}-Ph—(CH₂)n_{c}- and n_{c} represents an integer of 2 to 18, preferably 2 to 10, Rd represents —CH₂— or —SO₂—, Z¹ represents —O— or —NH—, and Ph represents (o-, m- or p-) phenylene group.

4. The carrier for nucleic acid molecule delivery according to item 3 wherein the repeating unit (B) containing the sugar is represented by the general formula (III): 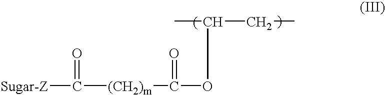
wherein Sugar represents a sugar residue obtained by removing one NH₂ from a sugar (in the cases of monosaccharide, disaccharide or polysaccharide where the sugar is bound at an amino group in amino sugar) or removing one OH from the sugar (in the cases of monosaccharide, disaccharide or polysaccharide where the sugar is bound at a hydroxyl group in saccharide), m represents an integer of 2 to 10, and Z represents O or NH.

5. The carrier for nucleic acid molecule delivery according to any of items 1 to 3 wherein the repeating unit (A) having the cationic group is a repeating unit represented by the following general formula (IV): 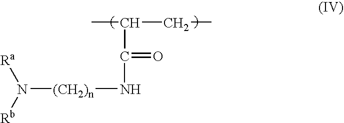
wherein n represents an integer of 1 to 10, and Rₐ and R_{b} may be the same or different and represents alkyl groups having 1 to 4 carbon atoms.

6. The carrier for nucleic acid molecule delivery according to any of items 2 to 4 wherein the repeating unit (C) having the hydrophobic substituent is a repeating unit represented by the following general formula (V): 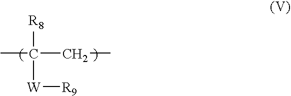
wherein R₈ represents —H or —CH₃; W represents —C(═O)O—, —OC(═O)—, —OC(═O)—(CH₂)n_{w}-C(═O)O— or —C(═O)NH and n_{w} represents an integer of 2 to 18, preferably 2 to 10; and R₉ represents a saturated or unsaturated aliphatic or alicyclic hydrocarbon group having 3 to 30 carbon atoms.

7. The carrier for nucleic acid molecule delivery according to any of items 2 to 6 wherein a molar ratio of the repeating unit (A)+the repeating unit (B) to the repeating unit (C) in the saccharified copolymer is A+B:C=99.9:0.1 to 0.1:99.9.

8. The carrier for nucleic acid molecule delivery according to any of items 1 to 7 wherein a weight average molecular weight of the saccharified copolymer is 10,000 to 1,000,000.

9. A transfection reagent or a carrier for gene therapy using the carrier for nucleic acid molecule delivery according to any of items 1 to 8.

10. A method of introducing a DNA into a cell characterized by applying the carrier for nucleic acid molecule delivery according to any of items 1 to 9 and the DNA to the cell.

11. The method according to item 10 characterized in that the above carrier contains a sugar residue and the above cell has a receptor for the sugar residue, and a complex of the above carrier and the DNA is incorporated into the cell by being mediated via the receptor.

### EFFECTS OF THE INVENTION

As is shown in the following results, it has been revealed that the carrier for nucleic acid molecule delivery formed from the saccharified copolymer of the present invention exhibits the high gene expression efficiency.

The carrier for nucleic acid molecule delivery of the present invention can suitably introduce a nucleic acid molecule into the cell depending on an internalization mechanism of the cell, and is particularly suitable for the nucleic acid molecule delivery by endocytosis mediated via the receptor.

The complex (of the nucleic acid molecule and the saccharified copolymer) internalized by endocytosis mediated via the receptor migrates rapidly into a nucleus, and the nucleic acid molecule is efficiently delivered into the nucleus by the carrier for nucleic acid molecule delivery of the present invention.

The carrier for nucleic acid molecule delivery of the present invention can be used suitably as particularly the carrier for delivery of a gene. The gene carried by the carrier for nucleic acid molecule delivery of the present invention is delivered into a cell, and subsequently is expressed at high efficiency. In particular, in the carrier for nucleic acid molecule delivery of the present invention formed from the saccharified copolymer having the repeating unit having the hydrophobic substituent, a DNA dissociation ability is high, and the gene internalized in the cell can be efficiently expressed.

The carrier for nucleic acid molecule delivery of the present invention comprises such excellent properties, and can be effectively utilized for gene transfection into various cells.

### BRIEF DESCRIPTION OF THE DRAWINGS

FIG. 1 is a drawing showing results of examining a relationship between an expression efficiency of luciferase and a C/A ratio in a complex of pCMV-Luc with a saccharified copolymer in HepG2 cells (A) and a complex of pCMV-Luc with a saccharified copolymer in COS cells (B). PEI indicates polyethyleneimine.

FIG. 2 is a drawing showing results of studying toxicity of polymers for cells by quantifying proteins by a DC protein method.

FIG. 3 is a drawing showing results of examining effects of galactose on gene expression efficiency in HepG2 cells. Poly-Gal, Poly-glu and PEI indicate the cases of using a galactose-containing copolymer, a glucose-containing copolymer and polyethyleneimine, respectively.

FIG. 4 is a drawing showing results of examining DNA uptake into HepG2 cells by Poly(DMAPAA-co-6-O-vinyladipoyl-D-galactose) and effects of galactose thereon. (A) and (B) indicate DNA uptake in nuclei and cytoplasm, respectively. In FIG. 4, sugar indicates galactose.

FIG. 5 is a drawing showing results of examining DNA uptake into HepG2 cells by Poly(DMAPAA-co-6-O-vinyladipoyl-D-glucose) and effects of galactose thereon. (C) and (D) indicate DNA uptake in nuclei and cytoplasm, respectively. In FIG. 5, sugar indicates glucose.

FIG. 6 is a drawing showing a ratio of intranuclear mobility of a gene transfected using Poly(DMAPAA-co-6-O-vinyladipoyl-D-galactose) or Poly(DMAPAA-co-6-O-vinyladipoyl-D-glucose) in HepG2 cells.

FIG. 7 is a drawing represented by modeling dissociation of a DNA from a polyplex by anion molecules.

FIG. 8 is a drawing showing a relationship between an expression efficiency of luciferase and a C/A ratio in HepG2 cells when complexes of pCMV-Luc with various saccharified copolymers were used.

FIG. 9 is a drawing for dissociation of a gene from a complex of a galactose-containing copolymer with pCMV-Luc in the presence of PVS.

FIG. 10A shows enzymatic study of cholesteryl ester synthesis.

FIG. 10B shows results of studying concentrations of DVA (divinyl adipate) in cholesteryl ester synthesis.

FIG. 11 shows AFM images of a polymer DNA complex depending on stearyl group (S) contents and identification of DNA dissociation by adding an anion polymer (PVSK) using agarose electrophoresis.

FIG. 12 shows identification of DNA dissociation in cells by FRET analysis. In FIG. 12, Gal-D-A-S0(a) and Gal-D-A-S2-10(b) indicate a polymer containing no stearyl group and a polymer containing 2 to 10% stearyl group, respectively. Cells: HepG2 cells, Excitation wavelength=495 nm, Bar=20 μm.

### BEST MODES FOR CARRYING OUT THE INVENTION

The carrier for nucleic acid molecule delivery of the present invention is formed from the saccharified copolymer further having the repeating unit (C) having the hydrophobic substituent in addition to the repeating units (A) and (B).

Repeating Unit (A) Having Cationic Group

A structure of the repeating unit having the cationic group can be optionally set up as desired, but in the present invention, it is preferable to be the structure represented by the following general formula (I): 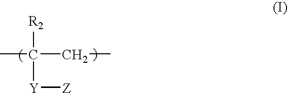
wherein R₂ represents H or CH₃; Y represents —C(═O)O—(CH₂)n_{y}-, —OC(═O)—(CH₂)n_{y}-, —OC(═O)—(CH₂)n_{y}-C(═O) or —CONH—(CH₂)n_{y}- and n_{y} represents an integer of 1 to 10; and Z represents —NR₃R₄ (R₃ and R₄ are the same or different and represent hydrocarbon groups having 1 to 10 carbon atoms), —N⁺R₅R₆R₇ (R₅, R₆ and R₇ are the same or different and represent hydrocarbon groups having 1 to 10 carbon atoms) or a nitrogen-containing heterocyclic group.

In one preferable embodiment of the present invention, the repeating unit (A) having the cationic group is represented by the following general formula (IV): 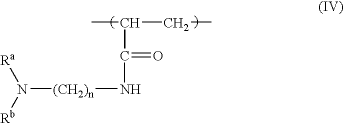
wherein R^{a}, R^{b} and n are the same as defined above.

Hydrocarbon groups represented by R₃ to R₇ include straight or branched saturated hydrocarbon groups or unsaturated hydrocarbon groups, or cyclic hydrocarbon groups. The cyclic hydrocarbon groups include aromatic hydrocarbon groups and alicyclic hydrocarbon groups. The hydrocarbon groups specifically include methyl, ethyl, propyl and butyl groups. R₃ and R₄ or R₅ and R₆ can come together with a nitrogen atom to which they are bound to form a pyrrolidine ring or a piperidine ring.

Nitrogen-containing heterocyclic groups include, for example, pyrrolidinyl, piperidinyl, piperidino, piperazinyl, N-methylpiperazino, morpholino, pyrrolyl, imidazolyl, pyridyl, pyrimidinyl, imidazolidinyl, quinolyl and isoquinolyl.

As alkyl groups having 1 to 4 carbon atoms represented by R^{a} and R^{b}, the alkyl groups having a straight chain or a branched chain such as methyl, ethyl, n-propyl, isopropyl, n-butyl, isobutyl, sec-butyl, t-butyl and the like are exemplified.

The repeating unit can be obtained by polymerizing a monomer having the cationic group.

The monomer having the cationic group include, for example, CH₂═CH—C(═O)O—CH₂)₃—N(CH₃)₂, CH₂═CH—C(═O)O—CH₂)₃—N⁺(CH₃)₃, CH₂═CH—OC(═O)—CH₂)₃—N(CH₃)₂, CH₂═CH—OC(═O)—CH₂)₃—N⁺(CH₃)₃, CH₂═CH—CONH—CH₂)₃—N(CH₃)₂, CH₂═CH—OC(═O)—CH₂)₃—C(═O)O—N(CH₃)₂ and CH₂═CH—OC(═O)—CH₂)₃—C(═O)O—N(CH₂CH₃)₂

Repeating Unit (B) Containing Sugar

The structure of the repeating unit containing the sugar can be optionally set up as desired, but in particular, the structure represented by the following general formula (II): 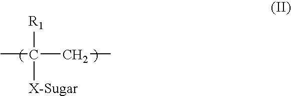
wherein R₁ represents H or CH₃; Sugar represents a sugar residue obtained by removing one NH₂ from a sugar (in the cases of monosaccharide, disaccharide or polysaccharide where the sugar is bound at an amino group in amino sugar) or removing one OH from the sugar (in the cases of monosaccharide, disaccharide or polysaccharide where the sugar is bound at a hydroxyl group in saccharide); and X represents —C(═O)O—, —C(═O)O—Rₐ—, —CONH—, —CONH—R_{b}—, —OC(═O)—Rₐ—C(═O)O— or —Ph—R_{d}—O— wherein Rₐ represents —Ph—O— or —(CH₂)nₐ-O— and nₐ represents an integer of 1 to 10, R_{b} represents —Ph—O— or —(CH₂)n_{b}-O— and n_{b} represents an integer of 1 to 10, R_{c} represents —(CH₂)n_{c}- or —(CH₂)n_{c}-Ph—(CH₂)n_{c}- and n_{c} represents an integer of 2 to 18, R_{d} represents —CH₂— or —SO₂—, and Ph represents phenylene group, is suitably used.

In general formula (II), “Sugar” represents the sugar residue obtained by removing one NH₂ (in the case of amino sugar) or one OH (in the case of greater parts of sugars other than amino sugar) from saccharide (monosaccharide, disaccharide, polysaccharide). When the sugar residue (Sugar) is bound at OH group, in the case of hexose, the sugar residue is bound to an anomeric carbon (position 1) or a hydroxyl group at position 2 or a primary hydroxyl group at position 6. In the case other than hexose, the sugar residue is bound to a position corresponding thereto. When the sugar residue is bound at NH₂ group, for example, in glucosamine, galactosamine and mannosamine, the carbon to which the NH₂ group has been bound is typically at position 2.

Sugar includes both saccharide represented by Sugar-(OH) and amino sugar represented by Sugar-(NH₂).

Types of the saccharide represented by Sugar-(OH) are not particularly limited, and includes monosaccharides such as glucose, fructose, mannose, galactose, xylose, erythrose, sorbose, ribose, ribulose and xylulose, disaccharides such as sucrose, maltose, cellobiose, agarobiose, isomaltose, xylobiose, gentinobiose, kojibiose, soforose, thalanose and trehalose, and polysaccharides such as maltotriose, raffinose, lacto-N-tetraose, dextrin, amylose, amylopectin, chitosan, starch, cellulose, α-cyclodextrin, β-cyclodextrin and γ-cyclodextrin, and hydrolytic products of polysaccharides. They may be natural saccharides or synthesized saccharides.

As the amino sugar represented by Sugar-(NH₂), monosaccharides such as glucosamine, galactosamine, mannosamine and neuraminic acid or disaccharides or polysaccharides having these amino monosaccharides are exemplified.

The type of saccharide can be optionally set up depending on the type of a biological sample to which the carrier for nucleic acid molecule delivery is applied. For example, when the gene is introduced into the cells such as hepatic parenchymal cells on which galactose receptors are present, galactose is suitably used.

The repeating unit (B) containing the sugar can be obtained by polymerizing a corresponding monomer containing the sugar.

The preferable monomer containing the sugar include, for example, vinyladipoyl-D-galactose, vinyladipoyl-D-glucose, acryloyl galactose, acryloyl glucose, vinylsebacyl glucose and adipoyl mannose.

Repeating Unit (C) Having a Hydrophobic Substituent

The structure of the repeating unit having the hydrophobic substituent can be optionally set up as desired, but in particular, the structure represented by the following formula (V): 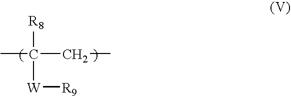
wherein R₈ represents —H or —CH₃; W represents —C(═O)O—, —OC(═O)—, —OC(═O)—(CH₂)n_{w}-C(═O)O— or —C(═O)NH and n_{w} represents an integer of 2 to 18; and R₉ represents a hydrocarbon group having 3 to 30 preferably 5 to 30 and more preferably 10 to 30 carbon atoms, is suitably used.

The hydrocarbon group of R₉ includes straight or branched saturated hydrocarbon groups (alkyl groups of C3 to C30 such as propyl, n-butyl, isobutyl, t-butyl, pentyl, hexyl, octyl, decyl, dodecyl, tetradecyl, hexadecyl, octadecyl (stearyl) and eicosyl) or unsaturated hydrocarbon groups (alkenyl groups of C3 to C30 such as allyl, butenyl, pentenyl, hexenyl, octenyl, decenyl, docenyl, tetradecenyl, hexadecenyl, octadecenyl and eicosenyl), or cyclic hydrocarbon groups. The cyclic hydrocarbon group includes aromatic hydrocarbon groups (e.g., phenyl, xylyl, toluyl, naphthyl, anthranil, phenanthryl, etc.), and saturated or unsaturated alicyclic hydrocarbon groups (cyclopentyl, cyclohexyl, cyclohexenyl, cholesteryl, groups derived from phytosterol). Phytosterol includes stigmasterol, stigmastanol, sitosterol (α₁, β, γ) and campesterol. R⁹ preferably includes stearyl, palmityl, pentadecanoyl, heptadecanoyl and cholesteryl.

The repeating unit (C) having the hydrophobic substituent can be obtained by polymerizing a monomer having the hydrophobic substituent.

The monomer having the hydrophobic substituent includes acrylic acid ester of higher fatty alcohol having 10 to 20 carbon atoms, vinyl ester of higher fatty acid having 10 to 20 carbon atoms, and vinyl monomers having a cholesterol moiety or a phytosterol moiety. More specifically, it is possible to exemplify stearyl acrylate and the vinyl monomer having the cholesterol moiety represented by the following general formula (VI): 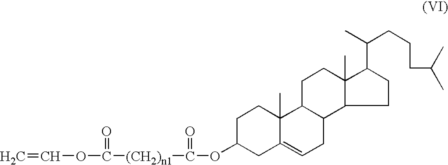
wherein n represents an integer of 2 to 18, preferably 2 to 10.

The above cholesterol derivative can be easily synthesized by a method of using an ester type polymerizable substituent and an enzyme.

As a solvent, an organic solvent such as tetrahydrofuran, toluene, pyridine and dioxane is used, and as the enzyme, lipase (LPL-311) derived from Pseudomonas sp or an immobilized enzyme (LIP-301) of LPL-311 is used. As the polymerizable substituent, divinyl dicarboxylate such as divinyl succinate, divinyl adipate and divinyl sebacate are exemplified. As with cholesterol, using the corresponding phytosterol, the corresponding phytosterol ester can be similarly synthesized. In general, ester of higher alcohol can also be similarly synthesized.

In vinyl ester used in the present invention, a polymer chain after the polymerization becomes polyvinyl alcohol. Polyvinyl alcohol has been used as a biomaterial since a long time ago, and is highly safe.

Saccharified Copolymer

The saccharified copolymer having the repeating unit (A) and the repeating unit (B) in the present invention can be obtained by copolymerizing a monomer having a cationic group and a monomer containing sugar. The copolymer containing the repeating unit (A), the repeating unit (B) and the repeating unit (C) in the present invention can be obtained by copolymerizing the monomer having the cationic group, the monomer containing the sugar and a monomer having a hydrophobic substituent.

As a method of copolymerizing respective monomers, it is possible to optionally used a publicly known method. For example, the polymerization can be performed by radical polymerization using a polymerization initiator.

As the polymerization initiator, it is possible to use a usual radical polymerization initiator, and for example, an azo based initiator such as azobisisobutyronitrile (AIBN), and an organic oxide can be used.

A molar ratio of the repeating units in the saccharified copolymer can be optionally set up for the purpose of obtaining the saccharified copolymer or the carrier for nucleic acid molecule delivery having the desired nature.

Typically, in the copolymer composed of the repeating unit (A) and the repeating unit (B), the molar ratio of (A) to (B) is A:B=about 1:99 to 99 to 1, preferably about 10:90 to 90:10, more preferably 15:85 to 50:50, and still more preferably 20:80 to 30:70.

In the case of having the repeating unit (C), the molar ratio of a summation of the repeating unit (A) and the repeating unit (B) to the repeating unit (C) is A+B:C=about 99.9:0.1 to 0.1 to 99.1, and preferably about 99.5:0.5 to 0.5 to 99.5. The ratio of the repeating unit (A) to the repeating unit (B) in the summation (A+B) of the repeating unit (A) and the repeating unit (B) is the same as the ratio of the above A to B in the copolymer composed of the repeating unit (A) and the repeating unit (B). More preferably, (A+B):C is 99.5:0.5 to 90:10, and still more preferably 99.3:0.7 to 95:5.

The molecular weight of the saccharified copolymer can be optionally set up as desired, and is typically about 10,000 to 1,000,000, preferably 15,000 to 50,000 and more preferably about 20,000 to 50,000 as a weight average molecular weight.

A repeating unit and a configuration unit other than the repeating units (A), (B) and (C) may be included in the saccharified copolymer of the present invention within the object of the present invention.

Carrier for Nucleic Acid Molecule Delivery

The carrier for nucleic acid molecule delivery of the present invention is formed using the above saccharified copolymer of the present invention.

The saccharified copolymer in the carrier for nucleic acid molecule delivery condenses a nucleic acid molecule by electrostatic interaction to form a complex with the nucleic acid molecule. This complex is internalized in a cell, and the nucleic acid molecule is delivered into the cell.

The complex of the saccharified copolymer with the nucleic acid molecule can be optionally formed by a publicly known method, and for example, the complex can be prepared by mixing a solution containing the nucleic acid molecule and a solution containing the saccharified copolymer.

Cells into which the nucleic acid is introduced include animal cells such as mammalian cells including human cells and insect cells, plant cells, fungal cells such as yeast cells and bacterial cells such as Escherichia coli cells, and are preferably the animal cells, particularly the mammalian cells.

Transfection into the cells can be carried out by allowing a cell transfection agent of the present invention and the nucleic acid molecule such as DNA and RNA to act upon the cells. The cell transfection agent and the nucleic acid molecule such as DNA and RNA may form the complex in advance, and subsequently the nucleic acid may be transfected by allowing the complex to act upon the cells.

The ratio of the saccharified copolymer to the nucleic acid molecule in the complex is represented by the ratio of a mole number (C) of the cationic group in the saccharified copolymer to a mole number (A) of phosphate group in DNA or RNA (hereinafter referred to as a C/A ratio), and is typically 0.5 or more, preferably 1.0 or more, and more preferably 1.5 or more.

It has been known that a phenomenon called endocytosis where a protein with high molecular weight is enfolded with a part of cell membrane to internalize the protein is present in the cell. In the endocytosis, there are liquid phase endocytosis with no specificity for a ligand and receptor mediated endocytosis (RME). The former is not useful as substance transport into the cell because it is non-specific and an internalization rate is slow whereas the latter recognizes the ligand at low concentration and effectively internalizes it into the cell, therefore, applicability of the carrier for nucleic acid molecule delivery by taking advantage of this mechanism is high. It has been known that RME is the mechanism which is present in many cells in liver, kidney, intestine, lung, muscle and placenta, adipose cells, erythrocytes and leukocyte fibers. The ligands are different depending on the respective cells, and galactose- and N-acetylgalactosamine-mediated internalization in hepatic parenchymal cells, and mannose-mediated internalization in hepatic non-parenchymal cells have been well-known. The carrier for nucleic acid molecule delivery of the present invention is particularly suitable for the nucleic acid molecule delivery by the endocytosis (RME) mediated via such a receptor.

The type of the nucleic acid molecule carried by the carrier for nucleic acid molecule delivery of the present invention is not particularly limited, and includes, for example, DNA or RNA. More specifically, genes, antisense DNA and plasmids encoding a particular protein, expression construct containing the gene, and iRNA are included.

The carrier for nucleic acid molecule delivery of the present invention can be suitably used particularly as the carrier for delivery of the gene. The gene carried by the carrier for nucleic acid molecule delivery of the present invention is delivered into the cell, and subsequently is expressed at high efficiency.

In particular, the copolymer having the hydrophobic repeating unit (C) is particularly preferable because the nucleic acid and the transfection agent are separated in the cell after being internalized into the cell, and the internalized nucleic acid is expressed at high efficiency.

### EXAMPLES

The present invention will be described more specifically using Examples, Experimental Examples and Comparative Examples, but the invention is not limited thereto.

### Example 1

### Gene Expression by Carrier for Nucleic Acid Molecule Delivery Formed Using poly(dimethylaminopropylacrylamide-co-6-O-vinyladipoil-D-galactose or poly(dimethylaminopropylacrylamide-co-6-o-vinyladipoil-D-glucose

(1-1) Synthesis of Saccharified Copolymer

Poly(dimethylaminopropylacrylamide-co-6-o-vinyladipoil-D-galactose was synthesized as follows.

A total amount was made 1 ml by using DMAPAA (dimethylaminopropylacrylamide) and 6-O-vinyladipoyl-D-galactose as monomers, and adding 1.0 mol % of 2,2-azobis(4-methoxy-2,4-dimethylbaleronitrile) (AMDVN) as an initiator and dimethylsulfoxide (DMSO) as a solvent. They were placed in a ground-glass-joint test tube with airtight stopper, frozen with liquid nitrogen after sealing with three way stopcock, and then nitrogen substitution was performed three times in the test tube. This was once again placed back to room temperature to melt, frozen again and the nitrogen substitution was repeated. This manipulation was repeated three times, and then without freezing, the test tube was directly deaerated using a vacuum line. Subsequently, the vacuumized test tube was immersed in a water bath at 60° C. to react for 4 hours. After 4 hours, a reactant was placed in a dialysis membrane with a molecular weight cutoff of 10,000, and dialyzed against purified water at room temperature for 24 hours. After 24 hours, the content in the dialysis membrane was placed in a glass tube, and lyophilized to yield polymer powder. A monomer ratio of DMAPAA to 6-O-vinyladipoyl-D-galactose to be added was 20:80, 35:65, 50:50, 65:35 or 80:20 (all units are mol %), and the same manipulation was given in each case.

The molecular weight, the molar ratio of the repeating units and the yield of the resulting galactose-containing copolymers are shown in the following Table 1. The molar ratio of the repeating units in the polymer was calculated by integral values obtained by ¹H-NMR measurement. ¹H-NMR measurement was performed by weighing 10 mg of the dried polymer, dissolving it in 700 μl of D₂O and using ¹H-NMR apparatus with 200 MHz. The weight average molecular weight of the polymer was measured by preparing a solution of the dried polymer at 5 mg/ml, and then performing GPC measurement using 1/15 M phosphate buffer (pH 7.42) as a mobile phase at a flow rate of 0.5 ml/min at a column temperature of 40° C., injecting 20 μl of a sample and using pullulan as a standard sample.

TABLE 1
Synthesis of poly(dimethylaminopropylacrylamide-co-6-o-
vinyladipoil-D-galactose

| | Added | | | |
|---|---|---|---|---|
| | ratio(mol %) | Molecular | Ratio(mol %) | |
| | DMAPAA:D- | weight | after polymerization | Yield |
| Sample | galactose | (×103) | DMAPAA:D-galactose | (%) |
| D28gal72 | 20:80 | 31 | 28:72 | 10 |
| D42gal58 | 35:65 | 30 | 42:58 | 21 |
| D56gal44 | 50:50 | 29 | 56:44 | 25 |
| D69gal31 | 65:35 | 26 | 69:31 | 33 |
| D80gal20 | 80:20 | 22 | 80:20 | 39 |

Glucose-containing copolymers were obtained by the same way in the above except that a glucose-containing monomer (6-O-vinyladipoyl-D-glucose) was used in place of the galactose-containing monomer.

The molecular weight and the molar ratio of the repeating units of the obtained glucose-containing copolymers are shown in the following Table 2.

TABLE 2
Synthesis of poly(dimethylaminopropylacrylamide-co-6-o-
vinyladipoil-D-glucose

| | Added | | | |
|---|---|---|---|---|
| | ratio(mol %) | Molecular | Ratio(mol %) | |
| | DMAPAA:D- | weight | after polymerization | Yield |
| Sample | glucose | (×103) | DMAPAA:D-glucose | (%) |
| D20glu80 | 20:80 | 32 | 20:80 | 27 |
| D40glu60 | 35:65 | 30 | 40:60 | 28 |
| D50glu50 | 50:50 | 29 | 50:50 | 30 |
| D66glu34 | 65:35 | 25 | 66:34 | 42 |
| D79glu21 | 80:20 | 22 | 79:21 | 39 |

(1-2) Formation and Identification of Complex of Nucleic Acid Molecule with Saccharified Copolymer

A plasmid, pCMV-Luc (Promega) was used as the nucleic acid molecule.

A stock solution was made by dissolving 2.4 mg of the saccharified copolymer made in the above (1-1) in 1000 μl of D-MEM medium. A C/A (cation/anion) ratio was made 50 by mixing 5 μl of this stock solution and 45 μl of pCMV-Luc solution (containing 100 ng of pCMV-Luc). Sample solutions with different C/A ratio (C/A ratios=0.5, 1, 1.5, 2, 3, 4 and 5) were prepared by serially diluting the stock solution and mixing it with the pCMV-Luc solution at the same concentration.

The formation of the complex of the saccharified copolymer and the nucleic acid molecule having the different C/A ratio was identified as follows by using 0.8% agarose gel electrophoresis. Using 0.5×TBE solution containing ethydium bromide (EtBr), 0.8% agarose gel was made. Subsequently, 10 μl of polymer (D40glu60, D42gal58) solution having C/A=0.5, 1, 1.5, 2, 3, 4 or 5, 1 μl of pCMV-Luc (200 ng/μl) and 2 μl of BPB solution were mixed to make 13 μl, and left stand at 37° C. for 30 minutes. After 30 minutes, the samples were applied in slots and electrophoresed at 100V for 30 minutes using 0.5×TBE with EtBr as a running buffer.

(1-3) Experiment for Gene Expression by Complex

A relationship between a gene expression efficiency and the C/A ratio in hepatic cells HepG2 cells and renal epithelial cells COS-1 cells was examined when pCMV-Luc-galactose-containing polymer complex and pCMV-Luc-glucose-containing polymer complex were used.

The gene expression efficiency was measured as follows.

As the carrier for nucleic acid molecule delivery, the complexes of the plasmid DNA (pCMV-Luc) and the saccharified copolymer (D40glu60, D42gal58) made in the above (1-2), i.e., the pCMV-Luc-galactose-containing polymer (poly(DMAPAA-co-6-O-vinyladipoyl-D-galactose) (D42gal58) complex and the pCMV-Luc-glucose-containing polymer (poly(DMAPAA-co-6-O-vinyladipoyl-D-glucose) (D40glu60)) complex were used.

As the cells into which the nucleic acid molecule was delivered, HepG2 cell which was a human hepatic cancer cell and COS-1 cell which was a grivet renal epithelial cell were used.

To COS-1cells and HepG2 cells seeded at 1×10⁴ cells/well in a 96-well plate and adhered thereto, 50 μl/well of the complex was added, 50 μl/well of 200 μM chloroquine solution (FBS free) was added thereto, and incubated for 8 hours. After 8 hours, the plate was washed with PBS, 100 μl/well of D-MEM (10% FBS) was added, and the plate was incubated for additional 40 hours. After 40 hours, the plate was washed with PBS, and 50 μl/well of a cell lysis solution (Triton X-100) was added. After being left stand at 37° C. for 30 minutes, 20 μl of cell lysate was taken out, and placed in a bottle in which 100 μl of a fluorescent substrate solution (ATP, D-luciferin, etc.) had been added. The gene expression efficiency in the cells was measured by, for example, a luciferase activity using a luminescence reader.

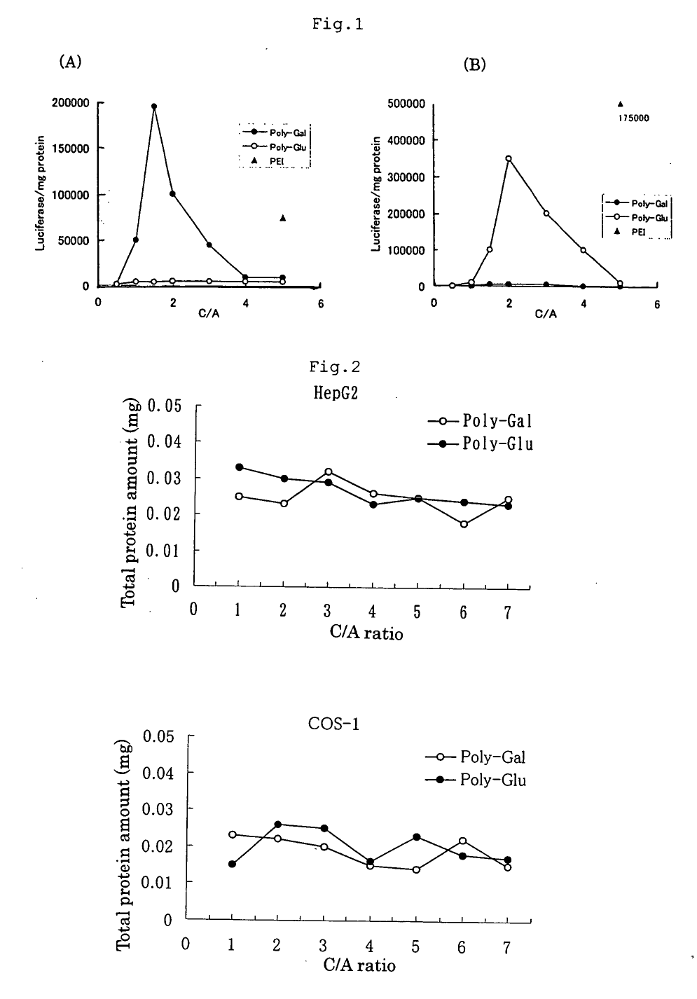

The result in the hepatic cells, HepG2 cells is shown in FIG. 1(A) and the result in the renal epithelial cells, COS-1 cells is shown in FIG. 1(B).

As a result, it has been revealed that the carrier for nucleic acid molecule delivery using poly(DMAPAA-co-6-O-vinyladipoyl-D-galactose) induces the high gene expression efficiency in the hepatic cells, HepG2 cells having the galactose receptor (FIG. 1(A)). The expression efficiency showed high values at C/A of 1.5 to 2. Meanwhile, in the result of the similar experiment using COS-1 cells, no facilitation of the internalization was observed (FIG. 1(B)). HepG2 cells have the galactose receptor whereas COS-1 cells have no galactose receptor. Therefore, it is conceivable that it is highly likely that poly(DMAPAA-co-6-O-vinyladipoyl-D-galactose) induced the gene expression specifically for the cells by being mediated via the receptor based on the galactose receptor.

On the contrary, the carrier for nucleic acid molecule delivery using poly(DMAPAA-co-6-O-vinyladipoyl-D-glucose) did not facilitate the internalization in HepG2 cells, but facilitated the internalization in COS-1 cells (FIGS. 1(A) and 1(B)).

(1-4) Evaluation of Toxicity for Cells

The toxicity for the cells was also evaluated by performing protein quantification by a DC protein method. The protein was quantified by Lowry method.

The results of comparing the toxicity of respective polymers [Poly(DMAPAA-co-6-O-vinyladipoyl-D-galactose) (D42gal58) and Poly(DMAPAA-co-6-O-vinyladipoyl-D-glucose) (D40glu60)] are shown in FIG. 2. When the cell died, a protein amount is decreased because the cell is fallen off from the plate. As shown in FIG. 2, a total protein amount was not changed in the experiment using any of the polymers. As a result, it has been conceivable that both polymers have almost no toxicity and there is no effect by the toxicity on the expression efficiency.

(1-5) Competitive Inhibition Experiment by Addition of Sugar

Then, in order to identify that the gene is incorporated by the mechanism mediated via the galactose receptor, a competitive inhibition experiment by the addition of sugar was performed by adding D-galactose in the gene expression experiment in the above (1-3).

If (poly(DMAPAA-co-6-O-vinyladipoyl-D-galactose) is internalized via the galactose receptor into HepG2 cells, D-galactose competes for binding to the galactose receptor with poly(DMAPAA-co-6-O-vinyladipoyl-D-galactose), and inhibits the binding of poly(DMAPAA-co-6-O-vinyladipoyl-D-galactose) to the receptor. Thus, it is conceivable that the gene expression efficiency in HepG2 cells induced by (poly(DMAPAA-co-6-O-vinyladipoyl-D-galactose) is reduced by the addition of D-galactose.

Poly(DMAPAA-co-6-O-vinyladipoyl-D-galactose) with C/A ratio of 2 which had exhibited the high gene expression efficiency was used for the experiment.

The competitive inhibition experiment by the addition of the sugar was performed as follows.

To COS-1cells and HepG2 cells seeded at 1×10⁴ cells/well in a 96-well plate and adhered thereto, 5 μl/well of D-galactose or D-glucose at various concentrations was added and 50 μl/well of the complex of the plasmid DNA (pCMV-Luc) and the polymer was added. Subsequently, 55 μl/well of 200 μM chloroquine solution (FBS free) was added thereto, and incubated for 8 hours. After 8 hours, the plate was washed with PBS, 100 μl/well of D-MEM (10% FBS) was added, and incubated for additional 40 hours. The gene expression efficiency was examined by measuring the luciferase activity.

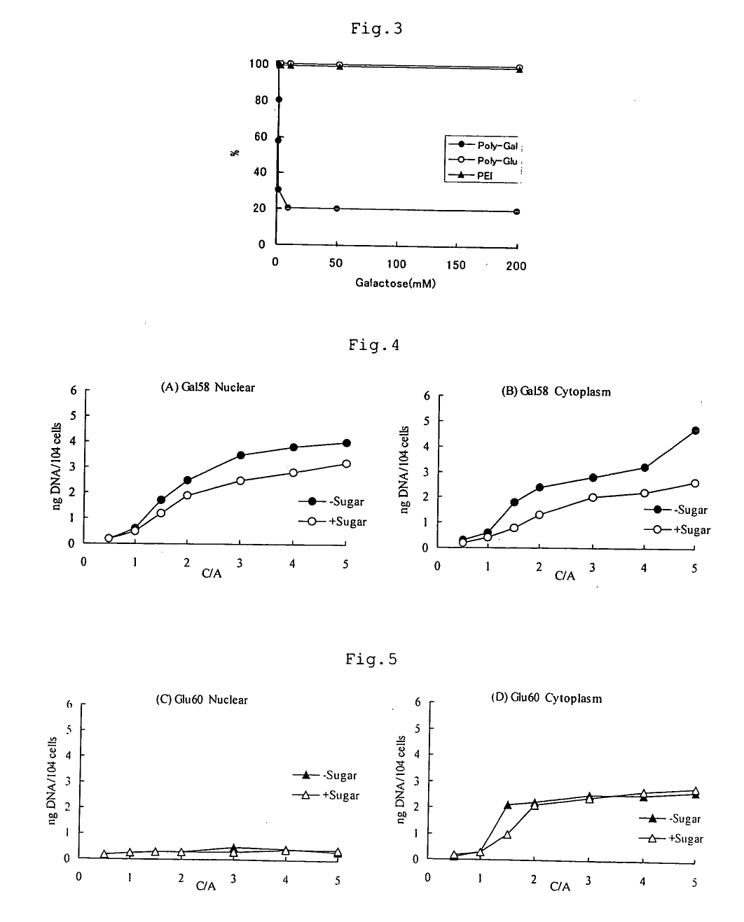

The results of the competitive inhibition experiment by the addition of galactose in the gene expression efficiency in HepG2 cells induced by poly(DMAPAA-co-6-O-vinyladipoyl-D-galactose) are shown in FIG. 3.

As a result, the gene expression efficiency in HepG2 cells induced by poly(DMAPAA-co-6-O-vinyladipoyl-D-galactose) was reduced to about 20% by the addition of galactose as shown in FIG. 3.

(1-6) Measurement of Location of Incorporated Gene by RI

Subsequently, in order to demonstrate whether the gene has been incorporated in nuclei or stayed in the cytoplasm in a mechanism of gene incorporation in the presence of the added sugar, ³²P-pCMV-Luc activity was measured in the competitive inhibition by the addition of the sugar to examine the incorporated amount in nuclei. The competitive inhibition by the addition of the sugar was performed by the same way as in the above (1-5).

### (1) Labeling of pCMV-Luc with ³²P

The plasmid pCMV-Luc was labeled with α-dCTP-³²P by a nick translation method.

### (2) Measurement of Incorporated ³²P-pCMV-Luc Amount

The incorporated amounts in the nuclei and the cytoplasm were determined by measuring radioactivity of ³²P-pCMV-Luc incorporated in the cells by a liquid scintillation counter. To COS-1cells and HepG2 cells seeded at 1×10⁴ cells/well in a 96-well plate and adhered thereto, D-galactose or D-glucose at various concentrations was added and 50 μl/well of the complex formed from ³²P-pCMV-Luc and the polymer (D40glu60, D42gal58) was added. Subsequently, 50 μl/well of 200 μM chloroquine solution (FBS free) was added thereto, and incubated for 8 hours. After 8 hours, the plate was washed with PBS, the cells were detached by adding a trypsin solution, and then lysed by adding an SDS solution. After centrifugation, a supernatant was separated from a precipitation, and each of them is placed in a liquid scintillation cocktail. The radioactivity of ³²P-pCMV-Luc incorporated into the cells was measured by the liquid scintillation counter. At that time, the activity in the supernatant corresponded to the incorporated amount in the cytoplasm and the activity in the precipitation corresponded to the incorporated amount in the nuclei.

The results of examining the gene incorporation into HepG2 cells by the complex using poly(DMAPAA-co-6-O-vinyladipoyl-D-galactose) (D42gal58) and the effect of galactose thereon are shown in FIG. 4.

As shown in FIG. 4, intranuclear incorporation of poly(DMAPAA-co-6-O-vinyladipoyl-D-galactose) was increased in HepG2 cells as the C/A ratio was increased. The incorporated amount in the cytoplasm was also increased as the C/A ratio was increased.

As shown in the experiment results in the above FIG. 1, the gene expression efficiency is maximized at C/A ratio of 1.5 to 2. On the contrary, as shown in the experiment results in FIG. 4, the amount of the gene incorporated into the cells is increased even at C/A>1.5. From these results, it has been thought that a simple correlation is not established between the expression efficiency and the incorporation of the complex of the polymer and the gene.

By adding D-galactose, the amount of incorporated ³²P-pCMV-Luc was reduced in the case of using poly(DMAPAA-co-6-O-vinyladipoyl-D-galactose).

This way, the result that the amount of the gene incorporated into the cells is reduced by adding galactose is thought to indicate that in the case of using poly(DMAPAA-co-6-O-vinyladipoyl-D-galactose), the gene is incorporated by the mechanism mediated via the galactose receptor.

The results of examining the DNA incorporation into HepG2 cells by the complex using poly(DMAPAA-co-6-O-vinyladipoyl-D-glucose) (D40glu60) and the effect of glucose thereon are shown in FIG. 5.

The incorporated amount of poly(DMAPAA-co-6-O-vinyladipoyl-D-glucose) in HepG2 cells was increased in the cells as the C/A ratio was increased, but the intranuclear incorporation exhibited low values.

The amount of incorporated ³²P-pCMV-Luc was scarcely changed by adding D-glucose in the case of using poly(DMAPAA-co-6-O-vinyladipoyl-D-glucose).

From these results, it has been thought that the gene incorporation in the case of using poly(DMAPAA-co-6-O-vinyladipoyl-D-glucose) is not attributed to the endocytosis mediated via the galactose receptor and occurs owing to the endocytosis which is non-specific incorporation in the cells.

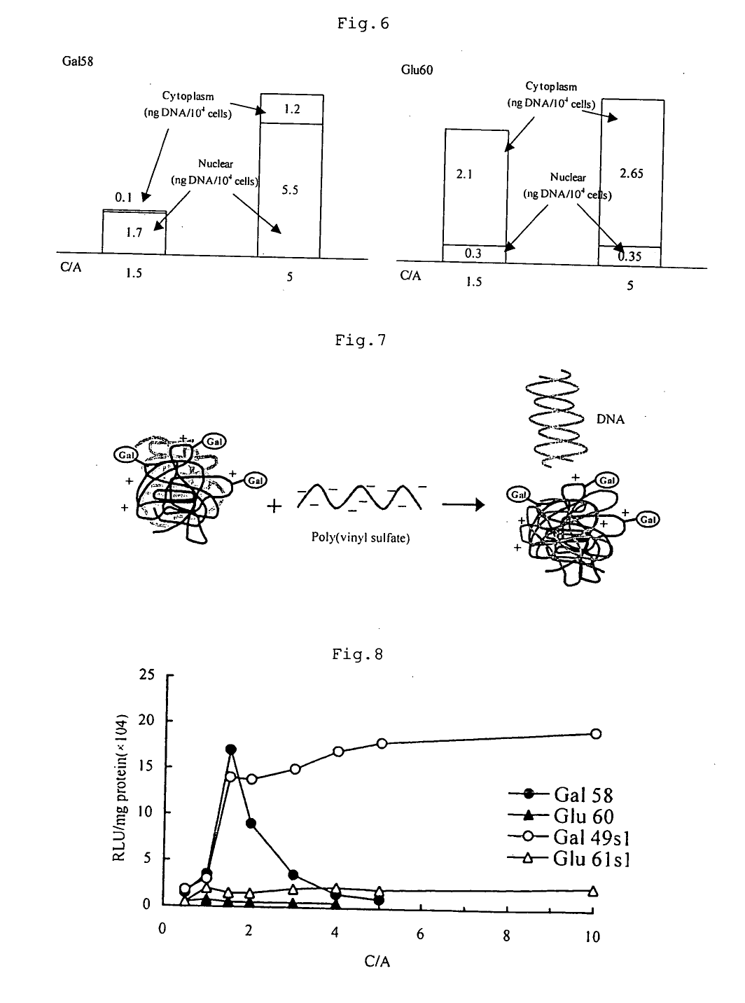

In order to elucidate the difference in incorporation mechanisms due to the type of the sugars, ratios of the amounts of the gene incorporated in the cytoplasm and the nuclei in the cases of using case of using poly(DMAPAA-co-6-O-vinyladipoyl-D-galactose) (D42gal58) and case of using poly(DMAPAA-co-6-O-vinyladipoyl-D-glucose) (D40glu60) were illustrated for the cases having the different C/A ratio in FIG. 6.

From these results, it has been found that a majority of the gene considered to be incorporated into the cytoplasm by D40glu60 not via the receptor stays in the cytoplasm regardless of difference in C/A ratio and an intranuclear mobility of the gene is low. Meanwhile, it has been found that a majority of the gene considered to be incorporated into the cytoplasm by D42gal58 via the galactose receptor migrates from the cytoplasm to the nucleus regardless of difference in C/A ratio. From these results, it has been thought that the complex incorporated by the endocytosis mediated via the receptor exhibits the high intranuclear mobility.

### Example 2

### Gene Expression by Carrier for Nucleic Acid Molecule Delivery Formed Using poly(DMAPAA-co-6-O-vinyladipoyl-D-glucose-co-stearyl or poly(DMAPAA-co-6-O-vinyladipoyl-D-galactose-co-stearyl)

It was thought that the gene expression efficiency was also increased along with the increase of the amount of the gene incorporated into the cells, but it has been revealed that the correlation is not observed between the amount of the incorporated gene and the gene expression efficiency as shown in the results in FIGS. 1 and 4. This has been thought to be likely caused because the incorporated amount is increased as the C/A ratio is elevated while DNA becomes difficult to be dissociated from the DNA-polymer complex (polyplex), thus the transcription and the translation become difficult and consequently the gene expression efficiency is also lowered. Thus, the dissociation of the DNA from the polyplex by anion molecules was studied (see FIG. 7). The change of the dissociation of the DNA from the polyplex was examined by synthesizing the copolymer using the monomer having a hydrophobic substituent to change a polymer shape.

That is, the correlation between the dissociation ability of the DNA from the polyplex by the anion molecules and the gene expression efficiency was compared by synthesizing the copolymers obtained by further introducing the repeating unit having stearyl group which was the hydrophobic substituent into the polymer having the cationic repeating unit and the saccharified repeating unit, specifically poly(DMAPAA-co-6-O-vinyladipoyl-D-glucose-co-stearyl and poly(DMAPAA-co-6-O-vinyladipoyl-D-galactose-co-stearyl), and using these polymers.

### (2-1) Synthesis of poly(DMAPAA-co-6-O-vinyladipoyl-D-glucose-co-stearyl and poly(DMAPAA-co-6-O-vinyladipoyl-D-galactose-co-stearyl)

Using DMAPAA as the monomer having the cationic group, 6-O-vinyladipoyl-D-glucose or 6-O-vinyladipoyl-D-galactose as the monomer containing the sugar, and stearylacrylate as the monomer having the hydrophobic substituent, the polymerization reaction was performed by the same method as in the synthesis of the copolymer in the Experimental Example. The concentration of the entire monomers was 0.5×10⁻³ mol, and the ratio of the monomers, DMAPAA:O-vinyladipoyl-D-glucose or 6-O-vinyladipoyl-D-galactose:stearylacrylate was 35:65:1 (all units are mol %). The initiator, 1.0 mol % of 2,2′-azobis(4-methoxy-2,4-dimethylvaleronitrile)(AMDVN) was added and DMSO was used as the solvent to make a total amount 1 ml. They were placed in a ground-glass-joint test tube with airtight stopper, frozen with liquid nitrogen after sealing with three way stopcock, and then nitrogen substitution was performed three times in the test tube. This was once again placed back to room temperature to melt, frozen again and the nitrogen substitution was repeated. This manipulation was repeated three times, and then without freezing, the test tube was directly deaerated using a vacuum line. Subsequently, the vacuumized test tube was immersed in a water bath at 60° C. to react for 4 hours. After 4 hours, a reactant was placed in a dialysis membrane with molecular weight cutoff of 10,000, and dialyzed against purified water at room temperature for 24 hours. After 24 hours, the content in the dialysis membrane was placed in a glass tube, and lyophilized.

The molecular weight, the molar ratio of the repeating units and the yield of the obtained polymers are shown in Table 3 for poly(DMAPAA-co-6-O-vinyladipoyl-D-galactose-co-stearyl) and Table 4 for poly(DMAPAA-co-6-O-vinyladipoyl-D-glucose-co-stearyl).

TABLE 3
Synthesis of poly(DMAPAA-co-6-O-vinyladipoyl-D-
galactose-co-stearyl)

| | Added ratio | | Molar ratio of | |
|---|---|---|---|---|
| | (mol %) | Mo- | repeating units after | |
| | DMAPAA:D- | lecular | polymerization (mol %) | |
| | galactose: | weight | DMAPAA:D-galactose: | Yield |
| Sample | Stearyl | (×10³) | stearyl | (%) |
| D50gal49sl | 35:65:1 | 29 | 50:49:1 | 23 |

TABLE 4
Synthesis of poly(DMAPAA-co-6-O-vinyladipoyl-D-glucose-
co-stearyl)

| | Added ratio | | Molar ratio of | |
|---|---|---|---|---|
| | (mol %) | Mo- | repeating units after | |
| | DMAPAA:D- | lecular | polymerization (mol %) | |
| | galactose: | weight | DMAPAA:D-galactose: | Yield |
| Sample | Stearyl | (×10³) | stearyl | (%) |
| D38glu61sl | 35:65:1 | 33 | 38:61:1 | 24 |

### (2-2) Identification of DNA Dissociation Ability

Potassium polyvinyl Sulfate Solution (PVSK) which was the anion molecule was added to the complex of poly(DMAPAA-o-6-O-vinyladipoyl-D-galactose-co-stearyl) (D50gal49s1) with the DNA and the complex of poly(DMAPAA-o-6-O-vinyladipoyl-D-glucose-co-stearyl) (D38glu61s1) with the DNA, and the DNA dissociation ability from the complex was identified by agarose gel electrophoresis. That is, 5 μl of the complex (C/A ratio=0.5, 1, 1.5, 2, 3, 4, 5, 10) of poly(DMAPAA-o-6-O-vinyladipoyl-D-galactose-co-stearyl) with the DNA (pCMV-Luc) or the complex (C/A ratio=0.5, 1, 1.5, 2, 3, 4, 5, 10) of poly(DMAPAA-o-6-O-vinyladipoyl- D-galactose) with the DNA (pCMV-Luc) was mixed with 1 μl of pCMV-Luc (200 μg/μl), and left stand at 37° C. for 30 minutes. Each 3.88 μl of PVSK at equivalent mol was added thereto, and the mixture was left stand at 37° C. for 30 minutes. Controls obtained by adding 5 μl of D-MEM and 3.88 μl of TE to 1 μl of pCMV-Luc (200 μg/μl) and adding 5 μl of the polymer solution at each C/A ratio and 3.88 μl of TE to 1 μl of pCMV-Luc (200 μg/μl) were also electrophoresed.

### (2-3) Measurement of Gene Expression Efficiency Using poly(DMAPAA-co-6-O-vinyladipoyl-D-galactose-co-stearyl) (D50gal49s1)

In order to identify the correlation between the DNA dissociation ability and the gene expression efficiency, gene transfection was performed using D50gal49s1. The complex of the plasmid DNA (pCMV-Luc) and the polymer was formed. To COS-1 cells and HepG2 cells seeded at 1×10⁴ cells/well in a 96-well plate and adhered thereto, 50 μl/well of the complex was added, 50 μl/well of 200 μM chloroquine solution (FBS free) was added, and incubated for 8 hours. After 8 hours, the plate was washed with PBS, 100 μl/well of D-MEM (10% FBS) was added, and incubated for additional 40 hours. After 40 hours, the plate was washed with PBS, and 50 μl/well of a cell lysis solution (Triton X-100) was added. After being left stand at 37° C. for 30 minutes, 20 μl of cell lysate was taken out, and placed in a bottle in which 100 μl of a fluorescent substrate solution (ATP, D-luciferin, etc.) had been added. The gene expression efficiency in the cells was measured by, for example, a luciferase activity using a luminescence reader.

Likewise, the gene expression efficiency was measured using the copolymers, poly(DMPAA-co-6-o-vinyladipoil-D-galactose) (D42gal58), poly(DMPAA-co-6-o-vinyladipoil-D-glucose) (D40glu60) and poly(DMAPAA-co-6-o-vinyladipoil-D-glucose-co-stearyl) (D38glu61s1).

Specifically, when the gene was transfected into HepG2 cells using the complex of pCMV-Luc with the copolymer of the above four types, the relationship between the expression efficiency of luciferase and the C/A ratio was examined. The results are shown in FIG. 8.

From the results shown in FIG. 8, it has been found that in the case of using poly(DMAPAA-co-6-O-vinyladipoyl-D-galactose) (D42Gal58), the intranuclear mobility is increased as the C/A ratio is elevated, but the expression efficiency attains to a peak at C/A ratio of 1.5.

Meanwhile, in the case of using poly(DMAPAA-co-6-O-vinyladipoyl-D-galactose-co-stearyl), as the C/A ratio was elevated, the amount of the gene expressed in HepG2 cells was increased.

From these results, when the gene is delivered using the galactose-containing copolymer into HepG2 cells, it has been suggested that the amount of the incorporated gene is increased as the C/A ratio is elevated, but the gene expression efficiency attains to the peak at C/A ratio of 1.5 when the DNA is difficult to be dissociated.

### (2-4) Dissociation of DNA by Addition of Anion Molecule

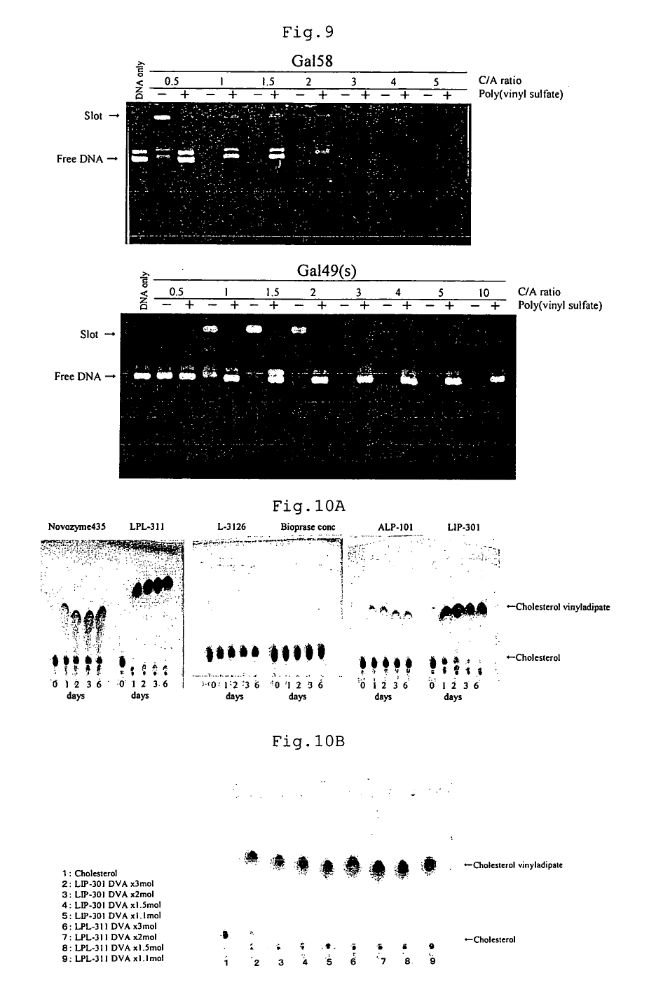

Furthermore, dissociation of the gene from the pCMV-Luc-Gal58 (pCMV-Luc-D42Gal58) complex and the pCMV-Luc-Gal49(s) (pCMV-Luc-D50Gal49s1) complex in the presence of PVS (Poly(vinylsulfate)) was examined. The dissociation of the gene was examined by the same way as in the above identification (2-2) of the DNA dissociation ability. The results are shown in FIG. 9.

When the C/A ratio was 1.5 using poly(DMAPAA-co-6-O-vinyladipoyl-D-glucose) (D42gal58), the DNA stayed in a slot in the absence of the anion molecule whereas the DNA was dissociated and bands which had run out from the slot could be identified in the presence of the anion molecule. When the C/A ratio was 2, the band of the DNA which had been dissociated and run out could be identified, but the band was much fainter than those when the C/A ratio was 1 or 1.5. When the C/A ratio was further elevated, the band of the DNA which had been dissociated and run out could not be identified. This suggests that the maximum expression efficiency at C/A ratio of 1.5 might be associated with the DNA dissociation ability. Meanwhile, in the case of using poly(DMAPAA-co-6-O-vinyladipoyl-D-galactose-co-stearyl) (D50gal49s1), even when the C/A ratio was 10, the DNA which had been dissociated and run out from the slot could be identified by adding the anion molecule.

From these results, if the DNA dissociation ability is associated with the gene expression efficiency, it is conceivable that by using poly(DMAPAA-co-6-O-vinyladipoyl-D-galactose-co-stearyl), the gene expression efficiency is also increased along with the elevation of the C/A ratio.

### Example 3

### Synthesis of Saccharified Copolymer

### (1) Methyl Galactoside-Containing Polymer Poly(DMAPAA-co-6-O-vinyladipoyl-methyl-D-galactoside-co-stearylacrylate)

DMAPAA (dimethylaminopropylacrylamide) as the monomer having the cationic group, 6-O-vinyladipoyl-methyl-D-galactoside as the monomer containing the sugar and stearylacrylate as the monomer having the hydrophobic substituent were used. The concentration of the entire monomers was 0.5×10⁻³ mol, and the ratio of cationic group:sugar:hydrophobic substituent to be added was 50:50:1 (all units are mol %). The initiator, 1 mol % of 2,2′-azobis(4-methoxy-2,4-dimethylvaleronitrile) (AMDVN) was added and DMSO was added as the solvent to make the total amount 1 ml in a glass ampoule. The ampoule was deaerated and sealed, and then the mixture was reacted at 60° C. for 24 hours. A reactant was placed in a dialysis membrane with molecular weight cutoff of 10,000, dialyzed against purified water for 24 hours, and then lyophilized. Resulting powder was washed with acetone and dried under vacuum to yield a polymer. The molecular weight, the molar ratio of the repeating units and the yield of the resulting polymer are shown in the following Table.

### Poly(DMAPAA-co-6-O-vinyladipoyl-methyl-D-galactoside-co-vinylstearate)

DMAPAA (dimethylaminopropylacrylamide) as the monomer having the cationic group, 6-O-vinyladipoyl-methyl-D-galactoside as the monomer containing the sugar and vinylstearate as the monomer having the hydrophobic substituent were used, and reacted by the same way as in the above. The molecular weight, the molar ratio of the repeating units and the yield of the resulting polymer are shown in the following Table.

### Poly(DMAPAA-co-6-O-vinyladipoyl-methyl-D-galactoside-co-vinylpalmitate)

DMAPAA (dimethylaminopropylacrylamide) as the monomer having the cationic group, 6-O-vinyladipoyl-methyl-D-galactoside as the monomer containing the sugar and vinylpalmitate as the monomer having the hydrophobic substituent were used, and reacted by the same way as in the above. The molecular weight, the molar ratio of the repeating units and the yield of the resulting polymer are shown in the following Table.

### Poly(DMAEA-co-6-O-vinyladipoyl-methyl-D-galactoside-co-stearyl)

DMAEA (dimethylaminoethylacrylate) as the monomer having the cationic group, 6-O-vinyladipoyl-methyl-D-galactoside as the monomer containing the sugar and stearylacrylate as the monomer having the hydrophobic substituent were used, and reacted by the same way as in the above. The molecular weight, the molar ratio of the repeating units and the yield of the resulting polymer are shown in the following Table.

### Poly(DMAEA-co-6-O-vinyladipoyl-methyl-D-galactoside-co-vinylstearate)

DMAEA (dimethylaminoethylacrylate) as the monomer having the cationic group, 6-O-vinyladipoyl-methyl-D-galactoside as the monomer containing the sugar and vinylstearate as the monomer having the hydrophobic substituent were used, and reacted by the same way as in the above. The molecular weight, the molar ratio of the repeating units and the yield of the resulting polymer are shown in the following Table.

### Poly(DMAEA-co-6-O-vinyladipoyl-methyl-D-galactoside-co-vinylpalmitate)

DMAEA (dimethylaminoethylacrylate) as the monomer having the cationic group, 6-O-vinyladipoyl-methyl-D-galactoside as the monomer containing the sugar and vinylpalmitate as the monomer having the hydrophobic substituent were used, and reacted by the same way as in the above. The molecular weight, the molar ratio of the repeating units and the yield of the resulting polymer are shown in the following Table.

### (2) Mannose-Containing Polymer

### Poly(DMAPAA-co-6-O-vinyladipoyl-D-mannose-co-stearylacrylate)

DMAPAA (dimethylaminopropylacrylamide) as the monomer having the cationic group, 6-O-vinyladipoyl-D-mannose as the monomer containing the sugar and stearylacrylate as the monomer having the hydrophobic substituent were used, and reacted by the same way as in the above. The molecular weight, the molar ratio of the repeating units and the yield of the resulting polymer are shown in the following Table.

### Poly (DMAPAA-co-6-O-vinyladipoyl-D-mannose-co-vinylstearate)

DMAPAA (dimethylaminopropylacrylamide) as the monomer having the cationic group, 6-O-vinyladipoyl-D-mannose as the monomer containing the sugar and vinylstearate as the monomer having the hydrophobic substituent were used, and reacted by the same way as in the above. The molecular weight, the molar ratio of the repeating units and the yield of the resulting polymer are shown in the following Table.

### Poly(DMAPAA-co-6-O-vinyladipoyl-D-mannose-co-vinylpalmitate)

DMAPAA (dimethylaminopropylacrylamide) as the monomer having the cationic group, 6-O-vinyladipoyl-D-mannose as the monomer containing the sugar and vinylpalmitate as the monomer having the hydrophobic substituent were used, and reacted by the same way as in the above. The molecular weight, the molar ratio of the repeating units and the yield of the resulting polymer are shown in the following Table.

### Poly(DMAEMA-co-6-O-vinyladipoyl-D-mannose-co-stearylacrylate)

DMAEMA (dimethylaminoethylmethacrylate) as the monomer having the cationic group, 6-O-vinyladipoyl-D-mannose as the monomer containing the sugar and stearylacrylate as the monomer having the hydrophobic substituent were used, and reacted by the same way as in the above. The molecular weight, the molar ratio of the repeating units and the yield of the resulting polymer are shown in the following Table.

### Poly(DMAEMA-co-6-O-vinyladipoyl-D-mannose-co-vinylstearate)

DMAEMA (dimethylaminoethylmethacrylate) as the monomer having the cationic group, 6-O-vinyladipoyl-D-mannose as the monomer containing the sugar and vinylstearate as the monomer having the hydrophobic substituent were used, and reacted by the same way as in the above. The molecular weight, the molar ratio of the repeating units and the yield of the resulting polymer are shown in the following Table.

### Poly(DMAEMA-co-6-O-vinyladipoyl-D-mannose-co-vinylpalmitate)

DMAEMA (dimethylaminoethylmethacrylate) as the monomer having the cationic group, 6-O-vinyladipoyl-D-mannose as the monomer containing the sugar and vinylpalmitate as the monomer having the hydrophobic substituent were used, and reacted by the same way as in the above. The molecular weight, the molar ratio of the repeating units and the yield of the resulting polymer are shown in the following Table.

### (3) Maltose-Containing Polymer

### Poly(DMAEA-co-6-O-vinyladipoyl-D-maltose-co-stearylacrylate)

DMAEA (dimethylaminoethylacrylate) as the monomer having the cationic group, 6-O-vinyladipoyl-D-maltose as the monomer containing the sugar and stearylacrylate as the monomer having the hydrophobic substituent were used, and reacted by the same way as in the above. The molecular weight, the molar ratio of the repeating units and the yield of the resulting polymer are shown in the following Table.

### Poly(DMAEA-co-6-O-vinyladipoyl-D-maltose-co-vinylstearate)

DMAEA (dimethylaminoethylacrylate) as the monomer having the cationic group, 6-O-vinyladipoyl-D-maltose as the monomer containing the sugar and vinylstearate as the monomer having the hydrophobic substituent were used, and reacted by the same way as in the above. The molecular weight, the molar ratio of the repeating units and the yield of the resulting polymer are shown in the following Table.

### Poly(DMAEA-co-6-O-vinyladipoyl-D-maltose-co-vinylpalmitate)

DMAEA (dimethylaminoethylacrylate) as the monomer having the cationic group, 6-O-vinyladipoyl-D-maltose as the monomer containing the sugar and vinylpalmitate as the monomer having the hydrophobic substituent were used, and reacted by the same way as in the above. The molecular weight, the molar ratio of the repeating units and the yield of the resulting polymer are shown in the following Table.

### (4) Trehalose-Containing Polymer

### Poly(DMAEMA-co-6-O-vinyladipoyl-D-trehalose-co-stearylacrylate)

DMAEMA (dimethylaminoethylmethacrylate) as the monomer having the cationic group, 6-O-vinyladipoyl-D-trehalose as the monomer containing the sugar and stearylacrylate as the monomer having the hydrophobic substituent were used, and reacted by the same way as in the above. The molecular weight, the molar ratio of the repeating units and the yield of the resulting polymer are shown in the following Table.

### Poly(DMAEMA-co-6-O-vinyladipoyl-D-trehalose-co-vinylstearate)

DMAEMA (dimethylaminoethylmethacrylate) as the monomer having the cationic group, 6-O-vinyladipoyl-D-trehalose as the monomer containing the sugar and vinylstearate as the monomer having the hydrophobic substituent were used, and reacted by the same way as in the above. The molecular weight, the molar ratio of the repeating units and the yield of the resulting polymer are shown in the following Table.

### Poly(DMAEMA-co-6-O-vinyladipoyl-D-trehalose-co-vinylpalmitate)

DMAEMA (dimethylaminoethylmethacrylate) as the monomer having the cationic group, 6-O-vinyladipoyl-D-trehalose as the monomer containing the sugar and vinylpalmitate as the monomer having the hydrophobic substituent were used, and reacted by the same way as in the above. The molecular weight, the molar ratio of the repeating units and the yield of the resulting polymer are shown in the following Table.

### (5) Glucose-Containing Polymer

### Poly(DMAPAA-co-6-O-vinyladipoyl-D-glucose-co-vinyladipoyl-cholesterol)

DMAPAA (dimethylaminopropylacrylamide) as the monomer having the cationic group, 6-O-vinyladipoyl-D-glucose as the monomer containing the sugar and 3-vinyladipoyl-cholesterol as the monomer having the hydrophobic substituent were used, and reacted by the same way as in the above. The molecular weight, the molar ratio of the repeating units and the yield of the resulting polymer are shown in the following Table.

3-Vinyladipoyl-cholesterol was synthesized as follows.

(1) Investigation of Solvents

An enzyme (LIP-301) (500 mg) was added to 5 ml of tetrahydrofuran in which 485 mg of cholesterol and 0.95 ml of divinyl adipate had been dissolved, and shaken at room temperature for 6 days. A reaction solution was developed on thin layer chromatography to identify the presence or absence of a product (hexane:ethyl acetate=9:1 as a development solvent, color was developed with sulfuric acid). The results are shown in Table 5.

[image]

(2) Investigation of Enzymes

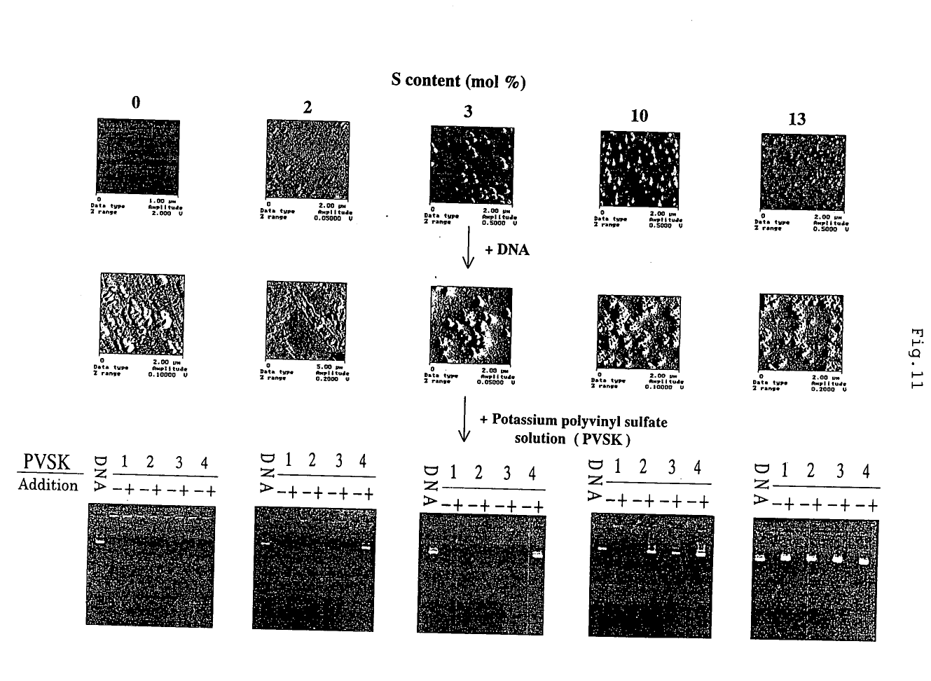

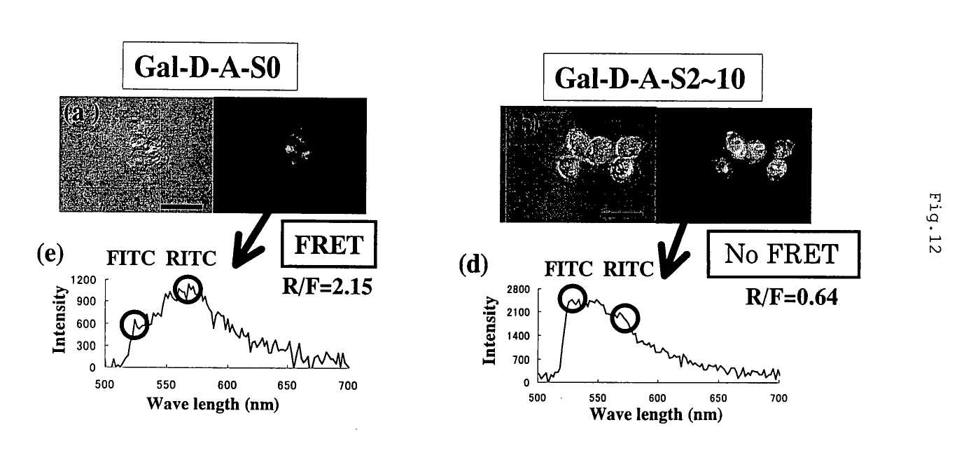

Each 5 ml of toluene, DMSO, DMF, pyridine, dioxane or tetrahydrofuran was added to 485 mg of cholesterol and 0.95 ml of divinyl adipate. As the enzyme, each 500 mg of lipase derived from Candida Antarctica: Novozyme435 (Novo), lipase derived from Pseudomonas sp, LPL-311 (Toyobo), the immobilized enzyme of LPL-311: LIP-301 (Toyobo), lipase derived from swine pancreas: L-3126 (Sigma), alkaline protease derived from Bacillus subtilis: Bioprase conc. (Nagase) or alkaline protease derived from Streptomyces sp.: ALP-101 (Toyobo) was added thereto, and the mixture was shaken at room temperature for 6 days. A reaction solution was developed on thin layer chromatography to identify the presence or absence of a product (hexane:ethyl acetate=9:1 as a development solvent, color was developed with sulfuric acid). The results are shown in FIG. 10A.

(3) Investigation of Divinyl Adipate Concentrations

The enzyme (LIP-301) (500 mg) was added to 5 ml of tetrahydrofuran in which 485 mg of cholesterol and divinyl adipate at 3 time mol, 2 time mol, 1.5 time mol or 1.1 time mol based on cholesterol had been added, and the mixture was shaken at room temperature for 6 days. A reaction solution was developed on thin layer chromatography to identify the presence or absence of a product (hexane:ethyl acetate=9:1 as a development solvent, color was developed with sulfuric acid). The results are shown in FIG. 10B.

(4) Scale Up Synthesis

Lipase (LIP-301) (2.3 g) was added to 23 ml of tetrahydrofuran in which 2.228 g of cholesterol and 4.36 ml of divinyl adipate had been dissolved, and the mixture was shaken at room temperature overnight. After the reaction, a reaction solution was filtrated to remove the insoluble enzyme, then the solvent was concentrated and applied on a column filled with 50 g of silica gel using hexane:ethyl acetate=20:1 as an eluate to yield 2.5 g of an oil product of ester which was a target.

TABLE 6
Synthesis of saccharified copolymer (added ratio [mol %] cationic
group:sugar:hydrophobic group = 50:50:1

| | | molecular | | |
|---|---|---|---|---|
| | | weight | Molar ratio after | |
| | Sample | (×10³) | polymerization* | Yield |
| Methyl | Poly(MeGal6VA-DMAEA-SA) | 16 | 76:21:3.1 | 53 |
| galactoside | Poly(MeGal6VA-DMAEA-SV) | 17 | 76:22:2.1 | 53 |
| | Poly(MeGal6VA-DMAEA-PV | 17 | 76:23:1.6 | 52 |
| | Poly(MeGal6VA-DMAPAA-SA) | 24 | 66:32:1.8 | 37 |
| | Poly(MeGal6VA-DMAPAA-SV | 24 | 68:31:0.9 | 42 |
| | Poly(MeGal6VA-DMAPAA-PV) | 24 | 67:32:0.9 | 48 |
| Mannose | Poly(Man-DMAAPAA-SA) | 19 | 52:47:1 | 20 |
| | Poly(Man-DMAAPAA-SV) | 17 | 48:51:0.9 | 38 |
| | Poly(Man-DMAAPAA-PV) | 18 | 45:53:1.2 | 39 |
| | Poly(Man-DMAEMA-SA) | 40 | 60:39:1 | 56 |
| | Poly(Man-DMAEMA-SV) | 47 | 60:39:0.3 | 60 |
| | Poly(Man-DMAEMA-PV) | 46 | 60:40:0.4 | 58 |
| Maltose | Poly(Mal-DMAEMA-SA) | 24 | 58:39:2.4 | 45 |
| | Poly(Mal-DMAEMA-SV) | 21 | 56:42:1.7 | 68 |
| | Poly(Mal-DMAEMA-PV) | 21 | 55:44:1 | 70 |
| Trehalose | Poly(Tre-DMAEMA-SA) | 31 | 67:33:0.4 | 71 |
| | Poly(Tre-DMAEMA-SV) | 32 | 67:32:0.4 | 65 |
| | Poly(Tre-DMAEMA-PV) | 33 | 69:31:0.3 | 74 |
| Glucose | Poly(Glu-DMAPAA-CholV) | 25 | 53:48:1 | 20 |

*cationic group:sugar:hydrophobic group (mol %)
SA: stearylacrylate,
SV: vinylstearate,
PV: vinylpalmitate,
CholV: vinyladipoyl-cholesterol

### Synthetic Example 1

### (1) Synthesis of monoacryloyl α-cyclodextrin by immobilized lipase

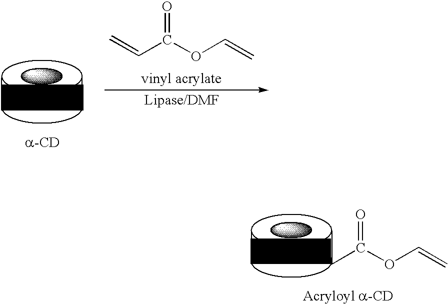

α-Cyclodextrin (Nacalai Tesque) (10 g) was dissolved in 50 ml of DMF, 1 g of immobilized lipase (LIP, Toyobo) and 8 g of molecular sieve 3A were added thereto, and the mixture was stirred for 2 hours. Vinyl acrylate (2 ml) was further added, and the mixture was stirred at room temperature for 48 hours. After the completion of the reaction, the reaction mixture was filtrated with High Flow Super Cell (supplied from Nacalai Tesque), further filtrated with a cartridge filter (MINISART SRP 15, supplied from Sartorius), and 300 ml of ethyl acetate was added to yield 5 g of a white precipitate of monoacryloyl α-cyclodextrin.

### (2) Synthesis of Poly(DMAPAA-co-acryloyl α-cyclodextrin-D-galactoside-co-stearyl)

DMAPAA (dimethylaminopropylacrylamide), monoacryloyl α-cyclodextrin and stearylacrylate were used. The concentration of the entire monomers was 0.5×10⁻³ mol, and the added ratio of cationic group:sugar:hydrophobic substituent was 50:50:1 (all units are mol %). The initiator, 1 mol % of 2,2′-azobis(4-methoxy-2,4-dimethylvaleronitrile) (AMDVN) was added and DMF was added as the solvent to make the total amount 1 ml in a glass ampoule. The ampoule was deaerated and sealed, and then the mixture was reacted at 60° C. for 24 hours. A reactant was placed in a dialysis membrane with molecular weight cutoff of 10,000, dialyzed against purified water for 24 hours, and then lyophilized. Resulting powder was washed with acetone and dried under vacuum to yield a polymer.

### Synthetic Example 2

### Synthesis of vinyladipoyl α-cyclodextrin by immobilized lipase

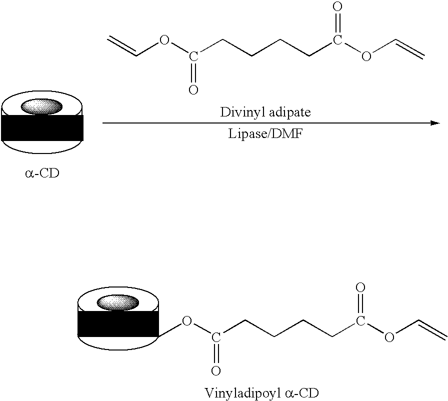

α-Cyclodextrin (Nacalai Tesque) (10 g) was dissolved in 50 ml of DMF, 1 g of immobilized lipase (LIP, Toyobo) and 8 g of molecular sieve 3A were added thereto, and the mixture was stirred for 2 hours. Divinyl adipate (2 ml) was further added, and the mixture was stirred at room temperature for 48 hours. After the completion of the reaction, the reaction mixture was filtrated with High Flow Super Cell (supplied from Nacalai Tesque), further filtrated with a cartridge filter (MINISART SRP 15, supplied from Sartorius), and 300 ml of ethyl acetate was added to yield 5 g of a white precipitate of vinyladipoyl α-cyclodextrin.

### (2) Synthesis of Poly(DMAPAA-co-vinyladipoyl α-cyclodextrin-D-galactoside-co-stearyl)

DMAPAA (dimethylaminopropylacrylamide), vinyladipoyl α-cyclodextrin and stearylacrylate were used. The concentration of the entire monomers was 0.5×10⁻³ mol, and the added ratio of DMAPAA:vinyladipoyl α-CD:stearylacrylate was 50:50:1 (all units are mol %). The initiator, 1 mol % of 2,2′-azobis(4-methoxy-2,4-dimethylvaleronitrile) (AMDVN) was added and DMF was added as the solvent to make the total amount 1 ml in a glass ampoule. The ampoule was deaerated and sealed, and then the mixture was reacted at 60° C. for 24 hours. A reactant was placed in a dialysis membrane with molecular weight cutoff of 10,000, dialyzed against purified water for 24 hours, and then lyophilized. Resulting powder was washed with acetone and dried under vacuum to yield a polymer.

### Test Example 1

Using RITC-labeled polymers (poly(DMAPAA-co-6-vinyladipoyl-D-galactoside-co-stearyl [S content was 0%]; Poly(DMAPAA-co-6-vinyladipoyl-D-galactoside-co-stearyl [S content was 2%]) having galactose containing stearyl group (S) (S contents: 0, 2, 3, 10, 13%) synthesized by the same way as in Example 3 as the sugar residue, the complex with the FITC-labeled DNA was formed. It was identified by allowing potassium polyvinyl sulfate salt (PVSK) to act upon the complex whether the DNA was dissociated or not. The results are shown in FIG. 11.

The polymer composed of only the cationic group containing no stearyl group as the hydrophobic group and the galactose group appeared to be in a straight chain shape because it was not identified as the AFM image (S content=0). At that time, the DNA dissociation was not observed in agarose gel electrophoresis even in the presence of the anionic polymer PVSK. Meanwhile, in the case of introducing 2 mol % or more stearyl group, both only the polymer and the formed DNA complex were identified to be in a particle shape by the AFM image. Furthermore, the phenomenon that the more the stearyl content was, the easier the DNA was to be dissociated was observed in the agarose gel electrophoresis.

The presence or absence of the DNA dissociation was identified by allowing the potassium polyvinyl sulfate (PVSK) salt to act upon the complex of this FITC-labeled DNA and the RITC-labeled polymer. The results are shown in FIG. 11 lower column. As shown in FIG. 11, it was identified that the more the stearyl group (S) content was, the easier the DNA dissociation was.

Furthermore, it was identified that the DNA was actually dissociated in the cell by FRET analysis of the gene transfection by the complex using HepG2 cell (FIG. 12). In the polymer (gal-D-A) containing no stearyl group, FRET was identified because fluorescence of RITC was increased when excited with excitation wavelength (495 nm) of FITC in the cell, indicating that the DNA and the polymer remained to form the complex in the cell. Meanwhile, in the polymer containing the stearyl group, the fluorescence of FITC was observed and no FRET occurred at excitation wavelength (495 nm) of FITC. Thus, this indicated that the DNA had been effectively dissociated from this polymer in the cell.

### INDUSTRIAL APPLICABILITY

By the use of the carrier for nucleic acid molecule delivery of the present invention, a nucleic acid can be effectively transfected into the cell, the transfected nucleic acid in the cell migrates in the nucleus, and the expression efficiency can be enhanced.

1. A carrier for nucleic acid molecule delivery comprising a saccharified copolymer having a repeating unit (A) having a cationic group, a repeating unit (B) containing sugar and a repeating unit (C) having a hydrophobic substituent.

2. The carrier for nucleic acid molecule delivery according to claim 1 wherein the repeating unit (A) having the cationic group is represented by the general formula (I): 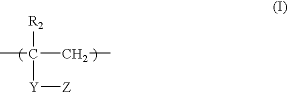
wherein R₂ represents H or CH₃; Y represents —C(═O)O—(CH₂)n_{y}-, —OC(═O)—(CH₂)n_{y}-, —OC(═O)—(CH₂)n_{y}-C(═O) or —CONH—(CH₂)n_{y}- and n_{y} represents an integer of 1 to 10; and Z represents —NR₃R₄ (R₃ and R₄ are the same or different and represent hydrocarbon groups having 1 to 10 carbon atoms), —N⁺R₅R₆R₇ (R₅, R₆ and R₇ are the same or different and represent hydrocarbon groups having 1 to 10 carbon atoms) or a nitrogen-containing heterocyclic group.

3. The carrier for nucleic acid molecule delivery according to claim 1 wherein the repeating unit (B) containing the sugar is represented by the general formula (II): 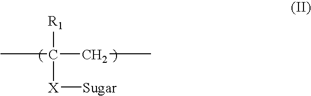
wherein R₁ represents H or CH₃; Sugar represents a sugar residue obtained by removing one NH₂ from a sugar (in the cases of monosaccharide, disaccharide or polysaccharide where the sugar is bound at an amino group in amino sugar) or removing one OH from the sugar (in the cases of monosaccharide, disaccharide or polysaccharide where the sugar is bound at a hydroxyl group in saccharide); and X represents —C(═O)Z¹, —C(═O)O—Rₐ—, —CONH—, —CONH—R_{b}—, —OC(═O)—R_{c}—C(═O)Z¹ or —Ph—R_{d}-Z¹ wherein Rₐ represents —Ph—O— or —(CH₂)nₐ-O— and nₐ represents an integer of 1 to 10, R_{b} represents —Ph—O— or —(CH₂)n_{b}-O— and n_{b} represents an integer of 1 to 10, R_{c} represents —(CH₂)n_{c}- or —(CH₂)n_{c}-Ph-(CH₂)n_{c}- and n_{c} represents an integer of 2 to 18, Rd represents —CH₂— or —SO₂—, Z¹ represents —O— or —NH—, and Ph represents (o-, m- or p-) phenylene group.

4. The carrier for nucleic acid molecule delivery according to claim 3 wherein the repeating unit (B) containing the sugar is represented by the following general formula (III): 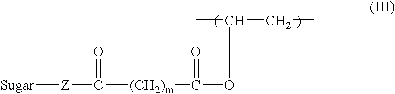
wherein Sugar represents a sugar residue obtained by removing one NH₂ from a sugar (in the cases of monosaccharide, disaccharide or polysaccharide where the sugar is bound at an amino group in amino sugar) or removing one OH from the sugar (in the cases of monosaccharide, disaccharide or polysaccharide where the sugar is bound at a hydroxyl group in saccharide), m represents an integer of 2 to 10, and Z represents O or NH.

5. The carrier for nucleic acid molecule delivery according to claim 1 wherein the repeating unit (A) having the cationic group is represented by the following general formula (IV): 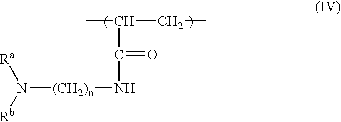
wherein n represents an integer of 1 to 10, and Rₐ and R_{b} may be the same or different and represent alkyl groups having 1 to 4 carbon atoms.

6. The carrier for nucleic acid molecule delivery according to claim 2 wherein the repeating unit (C) having the hydrophobic substituent is represented by the following formula (V): 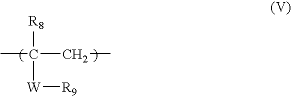
wherein R₈ represents —H or —CH₃; W represents —C(═O)O—, —OC(═O)—, —OC(═O)—(CH₂)n_{w}-C(═O)O— or —C(═O)NH and n_{w} represents an integer of 2 to 18; and R₉ represents a saturated or unsaturated aliphatic or alicyclic hydrocarbon group having 3 to 30 carbon atoms.

7. The carrier for nucleic acid molecule delivery according to claim 2 wherein a molar ratio of the repeating unit (A)+the repeating unit (B) to the repeating unit (C) in the saccharified copolymer is A+B:C=99.9:0.1 to 0.1:99.9.

8. The carrier for nucleic acid molecule delivery according to claim 1 wherein a weight average molecular weight of the saccharified copolymer is 10,000 to 1,000,000.

9. A transfection reagent or a carrier for gene therapy using the carrier for nucleic acid molecule delivery according to claim 1.

10. A method of introducing a DNA into a cell characterized by applying the carrier for nucleic acid molecule delivery according to any of claims 1 to 8 claim 1 and the DNA to the cell.

11. The method according to claim 10 characterized in that said carrier contains a sugar residue and said cell has a receptor for the sugar residue, and a complex of said carrier and the DNA is incorporated into the cell by being mediated via the receptor.
